(19) United States
(12) Patent Application Publication
(10) Pub. No.: US 2025/0017534 A1
(43) Pub. Date: Jan. 16, 2025

(54) Multimodal Cardiorespiratory Monitor

(71) Applicant: Mississippi State University, Mississippi State, MS (US)

(72) Inventor: Amirtaha Taebi, Mississippi State, MS (US)

(21) Appl. No.: 18/771,380

(22) Filed: Jul. 12, 2024

Related U.S. Application Data
(60) Provisional application No. 63/513,790, filed on Jul. 14, 2023.

(51) Int. Cl. A61B 5/00; A61B 5/0205; A61B 5/1455; A61B 5/352; A61B 7/04; G06N 3/08

(52) U.S. Cl. CPC A61B 5/7267; A61B 5/0205; A61B 5/14551; A61B 5/352; A61B 7/04; G06N 3/08; A61B 2562/0219

(57) ABSTRACT

A method and apparatus for classifying cardiorespiratory conditions using a multimodal approach and deep learning analysis. The method comprises receiving a training dataset of test subject records, each including a plurality of cardiorespiratory signal measurements and a corresponding diagnosis. The signal measurements are preprocessed to ensure common length and alignment with respect to cardiac and/or respiratory cycles, and features are extracted from the preprocessed signals. A deep learning model is trained on the features and diagnoses to classify cardiorespiratory signals into categories corresponding to different conditions. The apparatus comprises sensors configured to collect cardiorespiratory signals, a memory storing the trained deep learning model, and a processor. The processor preprocesses the signals, inputs them into the deep learning model, generates cardiorespiratory signal information, analyzes the information using the deep learning model to generate a classification result, and outputs the result, indicating the patient's cardiorespiratory health status.

### CROSS-REFERENCE TO RELATED APPLICATIONS

This application claims the benefit of U.S. Provisional Patent Application Ser. No. 63/513,790, filed Jul. 14, 2023, by Amirtaha Taebi, and entitled “Multimodal Cardiorespiratory Monitor,” which is hereby incorporated herein by reference in its entirety for all purposes.

### STATEMENT REGARDING FEDERALLY SPONSORED RESEARCH OR DEVELOPMENT

Not applicable.

### TECHNICAL FIELD

The present disclosure is generally related to the field of medical devices, and is specifically related to monitoring cardiorespiratory health using multiple physiological signals and machine learning analysis.

### BACKGROUND

Monitoring the health of the heart and lungs plays a role in the early detection and management of various cardiovascular and respiratory diseases. Traditional monitoring devices often rely on single physiological signals to assess specific aspects of heart and lung functions. These single-signal approaches may not provide a comprehensive understanding of the complex interactions and underlying mechanisms involved in heart and lung activity. Pulse oximeters, for example, measure blood oxygen levels but are known to have limitations in accuracy, particularly for individuals with specific congenital heart diseases or darker skin tones. This lack of accuracy can hinder the timely diagnosis and treatment of potentially life-threatening conditions. Furthermore, existing heart and lung-monitoring devices generally require patients to wear multiple sensors or undergo invasive procedures, which can be cumbersome, uncomfortable, and impractical for continuous monitoring in real-world settings.

### SUMMARY

In an embodiment, a method comprises receiving a training dataset comprising a plurality of test subject records, wherein each patient record comprises: a plurality of cardiorespiratory signal measurements from a patient; and a corresponding diagnosis of one or more cardiorespiratory conditions for the patient; preprocessing the plurality of cardiorespiratory signal measurements in each patient record to ensure a common length with cardiorespiratory cycles and a common alignment with cardiorespiratory cycles; training a deep learning model on cardiorepiratory data and corresponding diagnosis to classify cardiorespiratory signals into categories corresponding to cardiorespiratory conditions.

In an embodiment, a method comprises: collecting a plurality of cardiorespiratory signal measurements from a patient using a plurality of sensors; preprocessing the plurality of cardiorespiratory signal measurements to ensure a common length with respect to cardiorespiratory cycles and a common alignment with cardiorespiratory cycles using an electrocardiogram (ECG) signal and a seismocardiogram (SCG) signal as references; inputting cardiorepiratory data into a deep learning model trained to classify cardiorespiratory conditions; analyzing the cardiorepiratory data using the deep learning model to generate a classification result indicating a likelihood of a diagnosis of a cardiorespiratory condition; and outputting the classification result, wherein the classification result indicates a cardiorespiratory health status of the patient.

In an embodiment, an apparatus comprises: a plurality of sensors configured to collect a plurality of cardiorespiratory signal measurements; a memory configured to store a deep learning model trained on a dataset comprising a plurality of test subject records, each record including a plurality of cardiorespiratory signal measurements and a corresponding diagnosis of a cardiorespiratory condition; and a processor operatively coupled to the plurality of sensors and the memory, wherein the processor is configured to: preprocess the plurality of cardiorespiratory signal measurements to ensure a common length with respect to cardiorespiratory cycles and a common alignment with cardiorespiratory cycles using an ECG signal and a seismocardiogram (SCG) signal as a reference; input cardiorepiratory data into the deep learning model; analyze the cardiorepiratory data using the deep learning model to generate a classification result indicating a likelihood of a diagnosis of a cardiorespiratory condition; and output the classification result, wherein the classification result further indicates a cardiorespiratory health status.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1 is a block diagram illustrating the components and signal flow of a multimodal cardiorespiratory monitor in accordance with an embodiment of the disclosure.

FIG. 2 is a flowchart illustrating a method for training a deep learning model for classifying cardiorespiratory conditions in accordance with an embodiment of the disclosure.

FIG. 3 is a flowchart illustrating a method for monitoring cardiorespiratory conditions in a test subject using a multimodal cardiorespiratory monitor in accordance with an embodiment of the disclosure.

FIG. 4 is a diagram illustrating the placement of sensors on a human body for multimodal cardiorespiratory monitoring in accordance with an embodiment of the disclosure.

FIG. 5 is a schematic diagram of an example multimodal cardiorespiratory monitor in accordance with an embodiment of the disclosure.

### DETAILED DESCRIPTION

It should be understood at the outset that although an illustrative implementation of one or more embodiments are provided below, the disclosed systems and/or methods may be implemented using any number of techniques, whether currently known or yet to be developed. The disclosure should in no way be limited to the illustrative implementations, drawings, and techniques illustrated below, including the exemplary designs and implementations illustrated and described herein, but may be modified within the scope of the appended claims along with their full scope of equivalents.

Cardiorespiratory diseases are the leading cause of death in the United States and globally. Developing accurate and robust remote cardiorespiratory monitors may be useful. In addition to accuracy, practicality, and timeliness challenges associated with single-signal cardiorespiratory monitoring, there is a lack of existing devices capable of simultaneous, multi-signal data collection and analysis. While combining data from separate sensors post-hoc is theoretically possible, such an approach fails to capture the dynamic interplay between different physiological signals occurring within the same cardiac and/or respiration cycle. As a result, information may be lost or misrepresented, potentially leading to inaccurate diagnoses or missed opportunities for early intervention. The present disclosure addresses such limitations by introducing a multimodal cardiorespiratory monitor that combines multiple physiological signals and leverages deep learning and/or machine learning analysis to provide a more accurate and comprehensive assessment of cardiorespiratory health. It should be noted that while the present disclosure employs the term deep learning throughout the specification, other types of machine learning may also be used. Accordingly, the usage of the term deep learning should not be considered limiting unless context indicates otherwise.

By simultaneously collecting and analyzing a variety of cardiorespiratory signals, such as electrocardiograms (ECG), seismocardiogram (SCG), gyro cardiogram (GCG), phonocardiogram (PCG), pulse oximetry, body temperature, and chest impedance, the disclosure provides a more thorough understanding of underlying cardiovascular and respiratory processes.

Further, using deep learning algorithms allows for identifying subtle patterns and relationships within the multimodal data that may not be apparent with traditional analysis techniques. This can lead to the early detection of abnormalities and the prediction of potential cardiorespiratory events. Integrating deep learning into cardiorespiratory monitoring presents advancements over traditional methods. Deep learning models, with their abilities to learn complex patterns and relationships in large datasets, can uncover subtle indicators of cardiorespiratory dysfunction that may not be readily apparent. Developing effective deep learning models for cardiorespiratory health monitoring may require thorough consideration of data preprocessing, feature extraction, and model architecture to allow better performance and generalizability.

As can be seen, a more comprehensive, accurate, and non-invasive approach to cardiorespiratory health monitoring system that can capture a broader range of physiological signals and provide a more holistic assessment of cardiorespiratory function may be useful. Moreover, standardized datasets and evaluation metrics would facilitate comparing and validating different deep learning approaches in this field. The availability of such resources may accelerate the development and adoption of reliable and accurate deep learning models for cardiorespiratory health monitoring.

The present disclosure aims to address these challenges by providing a novel multimodal cardiorespiratory monitor that combines simultaneous data collection and deep learning analysis, along with user interfaces, to enhance the accuracy, comprehensiveness, and accessibility of cardiorespiratory health monitoring. The disclosure seeks to empower both healthcare providers and individuals with a powerful tool for early detection, diagnosis, and management of cardiorespiratory diseases, ultimately improving patient outcomes and quality of life.

This disclosure also aims to improve the accuracy and robustness of cardiorespiratory monitoring by mitigating the limitations of single-signal approaches, particularly for individuals with darker skin tones or specific congenital heart diseases. The present disclosure can also improve accuracy for monitoring of other types of cardiorespiratory conditions, such as heart failure. The combined analysis of multiple signals provides a more comprehensive picture of cardiorespiratory health, leading to more accurate diagnoses and personalized treatment plans.

Thus, the following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form, and some details of conventional elements may not be shown in interest of clarity and conciseness.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

The following acronyms, among others, may be used herein: cardiovascular disease (CVD), dynamic time warping (DTW), electrocardiogram (ECG), gyrocardiography/gyrocardiogram (GCG), inertial measurement unit (IMU), light-emitting diode (LED), microelectromechanical systems (MEMS), phonocardiography/phonocardiogram (PCG), photoplethysmography (PPG), seismocardiography/seismocardiogram (SCG), serial data (SDA), serial clock (SCL), and peripheral oxygen saturation (SpO2).

In the following discussion and in the claims, the terms “including” and “comprising” are used in an open-ended fashion and thus should be interpreted to mean “including, but not limited to . . . ” Also, the term “couple” or “couples” is intended to mean either an indirect or direct connection. Thus, if a first device couples to a second device, that connection may be through a direct engagement between the two devices or through an indirect connection that is established via other devices, components, nodes, and connections. In addition, as used herein, the terms “axial” and “axially” generally mean along or parallel to a particular axis (e.g., a central axis of a body or a port). In contrast, the terms “radial” and “radially” generally mean perpendicular to a particular axis. For instance, an axial distance refers to a distance measured along or parallel to the axis, and a radial distance means a distance measured perpendicular to the axis. As used herein, the terms “approximately,” “about,” “substantially,” and the like mean within 10% (i.e., plus or minus 10%) of the recited value. Thus, for example, a recited angle of “about 80 degrees” refers to an angle ranging from 72 degrees to 88 degrees.

The present disclosure relates to a multimodal cardiorespiratory monitor and associated methods for detecting and diagnosing cardiorespiratory conditions using a combination of physiological signals and deep learning analysis. The disclosure aims to provide a more comprehensive assessment of cardiorespiratory health compared to single-signal monitoring devices, enhancing early detection and diagnosis of various cardiorespiratory conditions.

Disclosed here is a multimodal cardiorespiratory monitor equipped with multiple sensors, each configured to collect a specific type of cardiorespiratory signal. These signals may include, but are not limited to, electrocardiograms (ECG) for electrical heart activity, seismocardiograms (SCG) for chest vibrations caused by heart activity (e.g., cardiovascular-induced axial vibrations of the chest), gyrocardiograms (GCG) (or gyroscopicardiograms) for torsional chest vibrations (e.g., cardiovascular-induced torsional vibrations of the chest), phonocardiograms (PCG) for heart sounds, pulse oximetry signals for blood oxygen levels, temperature signals for body temperature changes, and chest impedance signals for underlying tissue changes. The simultaneous collection of these diverse signals may allow for a more holistic understanding of the complex interplay between various physiological parameters influencing cardiorespiratory health. It should be noted that SCG, GCG, and PCG signals may provide information about both cardiovascular activity and respiratory activity. For example, SCG and GCG signals are filtered with appropriate digital filters to extract the chest vibrations due to respiration. The PCG signals can also be used to extract respiration sounds. These signals are beneficial for detecting respiratory diseases.

Simultaneous collection of signals may mean that the sensors operate concurrently to capture data within the same timeframe. Simultaneous data collection in this context may include targeted selection of signals, synchronization with cardiac and/or respiratory cycles, and integrated sensor design. Such synchronized data acquisition may aid in accurately analyzing the dynamic relationships between different physiological parameters and understanding their combined impact on cardiorespiratory health.

The collected signals may undergo preprocessing to improve accurate and meaningful analysis. This may involve filtering to remove noise and artifacts, aligning the signals based on cardiac cycles using the ECG signal as a reference or based on respiratory cycle using the low-frequency chest vibrations captured by SCG, and resampling to a common length with respect to an average cardiac and/or respiratory cycle. The resulting preprocessed signals may then be input into a deep learning model, which has been trained on a curated dataset comprising a plurality of test subject records. Each record may include a set of cardiorespiratory signal measurements along with a corresponding diagnosis of a cardiorespiratory condition.

By analyzing the preprocessed signals, the model may extract relevant features, including temporal, spectral, statistical, and morphological characteristics, that may serve as subtle indicators of cardiorespiratory dysfunction. A deep learning model, such as a convolutional neural network (CNN) or recurrent neural network (RNN), may be trained on the extracted features and corresponding diagnoses to learn the complex patterns and relationships that distinguish different cardiorespiratory conditions. These features may then be utilized to classify the test subject's cardiorespiratory status into various categories, ranging from a binary assessment of normal or abnormal to a more granular list of potential medical diagnoses.

The classification results, which may be accompanied by confidence scores or a list of potential diagnoses, may be presented to the user through an interface. This interface may include visual displays, mobile notifications (e.g., via mobile phone, watch, tablet, or computer), or audible alerts designed to facilitate more straightforward and prompt interpretation. The monitor's design may empower healthcare professionals and individuals to utilize this technology for early detection, diagnosis, and management of cardiorespiratory diseases, ultimately improving patient outcomes and overall well-being.

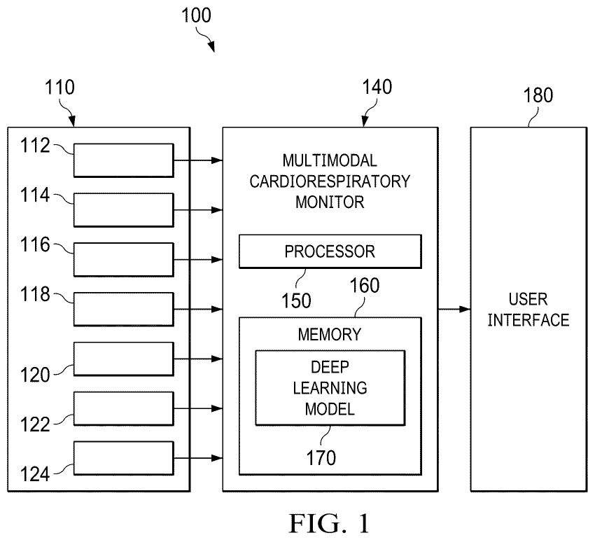

FIG. 1 is a block diagram depicting components and signal flow of multimodal cardiorespiratory monitor system 100 according to an embodiment of the disclosure. Multimodal cardiorespiratory monitor system 100 comprises signal inputs 110 (aka cardiorespiratory signal measurements 110), multimodal cardiorespiratory monitor 140, processor 150, memory 160, and user interface 180. A plurality of sensors may be configured to collect a plurality of cardiorespiratory signal measurements, such as signal inputs 110. Signal inputs 110 may be gathered from various sources, such as directly from a test subject. The depicted components are also powered by a power source, such as a battery, electrical plug and wire, etc.

As discussed for cardiorespiratory signal measurements in general, signal inputs 110 may be from various types of sensors. These sensors may be implemented external to multimodal cardiorespiratory monitor 140, more generally in multimodal cardiorespiratory monitor system 100 (as depicted), or within multimodal cardiorespiratory monitor 140. If incorporated into multimodal cardiorespiratory monitor 140, sensor placement may be considered to account for signal interference and data synchronization.

Signal inputs 110 may include signals from ECG sensor 112, SCG sensor 114, GCG sensor 116, PCG sensor 118, pulse oximeter 120, chest impedance sensor 122, body temperature sensor, or other useful sensors. ECG sensor 112 measures the heart's electrical activity, producing an electrocardiogram (ECG) signal. SCG sensor 114 may be a three-axis accelerometer used to capture vibrations in the X (i.e., right-left), Y (i.e., head-foot), and Z (i.e., back-front or dorsoventral) directions. SCG sensor 114 aids in detecting vibrations on a chest surface caused by heart and lung activity, generating an SCG signal. GCG sensor 116 measures the torsional vibrations on the chest surface due to heart and lung activity, producing a GCG signal. PCG sensor 118, using a microphone, captures heart and lung sounds, generating a PCG signal. Pulse oximeter 120 measures the blood oxygen saturation level, providing a pulse oximetry signal. Chest impedance sensor 122 measures changes in the electrical impedance of the chest, generating a chest impedance signal. Signal inputs 110 may be considered raw data.

Signals may be collected individually or simultaneously. Different cardiorespiratory signals provide unique and often complementary insights into cardiovascular and respiratory health. For instance, as discussed, ECG reveals electrical activity, SCG captures mechanical vibrations, pulse oximetry measures blood oxygenation, and so on. For simultaneous data collection, specific combinations, integrations, or configurations of multiple sensor types within a single device may exist. Combining these diverse signals may offer a more holistic and accurate assessment compared to relying on a single modality. The plurality of sensors may allow the detection of subtle patterns and correlations that single-signal devices may miss. This can lead to earlier and more accurate diagnoses of a broader range of cardiorespiratory conditions, including those that may manifest differently in various individuals.

In an embodiment, simultaneous data collection may also involve aligning signals with cardiac cycles, using an ECG signal as a reference or respiratory cycle using the low-frequency chest vibrations captured by SCG. This synchronization may aid in capturing dynamic interplays between different physiological parameters and more accurately extract features relevant to diagnosing specific cardiorespiratory conditions.

Memory 160 may be configured to store trained deep learning model 170, which may be used to classify cardiorespiratory conditions based on the input signals. Memory 160 can be any suitable type, such as flash memory, RAM, or a combination. Deep learning model 170 is an analytical tool that utilizes artificial intelligence to extract meaningful information from multimodal cardiorespiratory signals. Deep learning model 170 may be trained on a dataset comprising a plurality of test subject records, each record including a plurality of cardiorespiratory signal measurements and a corresponding diagnosis of a cardiorespiratory condition. Memory 160 may also store instructions for segmenting the signal inputs 110 based on cardiac cycles. Memory 160 may also store temporary data during processing.

The use of deep learning in medical diagnostics is an emerging field. Deep learning models, trained on large datasets, can identify patterns and correlations that are difficult for humans to discern, leading to improved diagnostic accuracy. The relationships between the various cardiorespiratory signals (e.g., ECG, SCG, GCG) and various conditions are complex and often nonlinear. Deep learning model 170, with its ability to learn intricate patterns from high dimensional data, may be well-suited to handle this complexity. Memory 160 may be readily accessible by processor 150, as deep learning model 170 may be loaded and executed during the analysis of incoming cardiorespiratory signals.

Processor 150 may be a microcontroller or central processing unit that is operatively coupled to sensors for signal inputs 110 and memory 160. Signals gathered from signal inputs 110 are received by processor 150. The collection of various signals may happen simultaneously or individually, depending on system requirements. Processor 150 may perform signal preprocessing tasks such as filtering, noise reduction, and alignment of signals with cardiac cycles. Alignment of signals with cardiac cycles may be done using an ECG as a reference. Also, alignment of signals with respiratory cycle may be done using the low-frequency chest vibrations captured by SCG That is, processor 150 may preprocess the plurality of cardiorespiratory signal measurements (e.g., signal inputs 110) to ensure a common length with cardiac cycles and a common alignment with cardiac cycles using an ECG signal as a reference.

Alignment of signals with cardiac and/or respiratory cycles may be useful because different sensors often operate at distinct sampling frequencies (e.g., ECG at 100 Hz, SCG at 50 Hz, ECG at 50 Hz, SCG at 100 Hz, or other frequencies depending on the sensors). Directly combining signals with varying lengths may lead to misalignment and misrepresentation of the temporal relationship between events in a cardiac cycle. Cardiorespiratory signals are generally intrinsically linked to the cardiac and/or respiratory cycle. Analyzing these signals in isolation or without proper alignment can obscure crucial information about their interdependence. Accurate feature extraction from the signals, which is usually used for subsequent deep learning analysis, relies on consistent signal lengths and alignment with cardiac phases (e.g., systole, diastole). The ECG signal, with its well-defined waveforms (e.g., P, QRS, T) corresponding to specific cardiac events, serves as a practical reference for signal alignment with the cardiac cycle. By identifying characteristic points in the ECG signal (e.g., R-peaks), the preprocessing step can more accurately segment and align other cardiorespiratory signals (e.g., SCG, GCG) to the corresponding cardiac and respiratory cycles. It should be noted that the low-frequency component of the SCG signals can be also used to align the signals with the respiratory cycle.

It should also be noted that there are other reasons for categorizing/aligning the signals with cardiac/respiratory cycles. For example, categorizing the signals using different respiratory phases can lead to an earlier diagnosis of particular cardiorespiratory conditions. For example, cardiac cycles that happen during inhalation may provide information different than those occurring during exhalation. This might be due to varying physiological conditions during these different respiratory phases, such as the pressure surrounding the heart muscle or intrapulmonary pressure.

Processor 150 may also generally handle the execution of the deep learning model for data analysis and classification. For example, processor 150 may input preprocessed cardiorespiratory signals into deep learning model 170, generate cardiorespiratory signal information, analyze the cardiorespiratory signal information using deep learning model 170 to generate a classification result indicating a likelihood of a diagnosis of a cardiorespiratory condition, and output the classification result indicating a cardiorespiratory health result. Cardiorespiratory health status may be indicated using a binary set of status groups comprising a “normal” cardiorespiratory status and an “abnormal” cardiorespiratory status. Alternatively, or in addition, cardiorespiratory health status may comprise a list of potential medical diagnoses. Thus, deep learning model 170 may be able to provide a more nuanced assessment, indicating the likelihood of specific cardiorespiratory conditions, such as atrial fibrillation, heart failure, sleep apnea, or comorbidity of two or more cardiorespiratory conditions. This information can be invaluable for early detection and personalized treatment planning.

User interface 180 may be incorporated within multimodal cardiorespiratory monitor 140 or be more generally within multimodal cardiorespiratory monitor system 100 external to the multimodal cardiorespiratory monitor 140 (as depicted in FIG. 1). User interface 180 may provide a visual or auditory output classification results to the user. User interface 180 may be a display screen on a wearable device, a mobile, watch, ring, or computer application, or audible alerts. User interface 180 may also provide additional information such as heart rate, heart rate variability, respiratory rate, blood oxygen level, or diagnosis confidence scores. User interface 180 may also include connectivity features for data transmission to healthcare professionals or cloud storage.

Simultaneous collection and analysis of multiple cardiorespiratory signals may allow for a more comprehensive and accurate assessment of cardiorespiratory health than single-signal monitoring devices. The integrated deep learning model may leverage this data to identify subtle patterns and correlations, potentially leading to early detection and improved diagnosis of cardiorespiratory conditions.

Furthermore, while deep learning models are often considered “black boxes” due to their complex internal workings, efforts may be made to enhance their interpretability in the medical field. This can involve techniques like attention mechanisms, which highlight the specific features or signal segments that contribute to the classification decision. Additionally, the model's output can be presented in a user-friendly manner, such as through visual explanations or clear probability scores, to aid clinicians in understanding the reasoning behind the diagnosis.

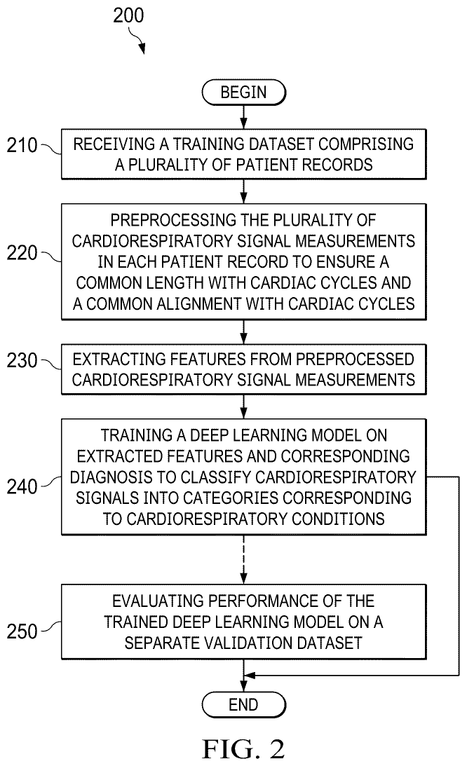

FIG. 2 illustrates a method 200 for training a deep learning model capable of classifying cardiorespiratory conditions based on multimodal analysis of various physiological signals according to an embodiment of the disclosure. The approach leverages a training dataset and signal processing techniques to enhance the accuracy and robustness of a classification model. The deep learning model employed in an embodiment may operate under a supervised learning paradigm. This means it learns to recognize patterns in data by being presented with examples where the correct answer (e.g., the diagnosis) is known.

At step 210, the method 200 comprises receiving a training dataset comprising a plurality of test subject records. Each record contains a collection of cardiorespiratory signal measurements, such as ECG, SCG, GCG, PCG, pulse oximetry, body temperature, and chest impedance signals (either individually or in combination), as well as a corresponding diagnosis of a cardiorespiratory condition for the test subject. This multimodal approach may provide a more comprehensive view of cardiorespiratory health compared to single-signal monitoring.

The training dataset can be seen as essentially serving as the teacher for the model. It provides a collection of real-world examples, allowing the model to learn the complex relationships between cardiorespiratory signals and various health conditions. Gathering from a plurality of test subject records may underscore the value of a diverse and representative dataset. The model's performance and generalizability may depend on its exposure to a wide range and/or quantity of cardiorespiratory signal patterns and associated diagnoses.

The training dataset may be assembled from various sources, such as clinical trials, hospital records, or specialized research studies. The training dataset may also include demographic information, such as age, gender, and ethnicity. This can enhance the model's ability to account for individual variations and improve its generalizability across diverse patient populations. Similarly, the training dataset may be further categorized based on specific types of cardiorespiratory conditions. By categorizing the training dataset based on specific cardiorespiratory conditions (e.g., atrial fibrillation, heart failure, hypertension), the deep learning model can be trained more effectively to recognize the unique signal patterns associated with each condition. General training on a mixed dataset might lead to a model that excels in overall classification but lacks the specificity to distinguish between closely related conditions.

Categorization may allow for targeted training on subsets of data, enhancing the model's ability to differentiate subtle variations in signals that are characteristic of specific diseases. Further, the training dataset may be categorized using other approaches such as diagnosis-based categorization, severity-based categorization, or subgroup analysis. Diagnosis-based categorization may be where the test subject records are grouped based on their confirmed medical diagnoses. For example, all records with a diagnosis of atrial fibrillation may be grouped together, as may those with heart failure, hypertension, and the like. In addition to diagnosis, the dataset can be further categorized based on the severity of the condition (e.g., severity-based categorization). This can help the model learn to distinguish between mild, moderate, and severe cases of a particular disease. The dataset can also be categorized based on specific subgroups of patients, such as age groups, genders, or ethnicities. This can enable the model to identify patterns that are unique to certain populations, leading to more personalized diagnoses.

Each patient record containing a plurality of cardiorespiratory signal measurements may mean that multiple types of signals (e.g., ECG, SCG, GCG) are collected simultaneously from each test subject. This multimodal approach may provide a more comprehensive picture of cardiorespiratory health than single-signal datasets. By associating specific signal patterns with confirmed diagnoses, the model may develop the ability to classify new, unseen signals accurately.

It may be beneficial to utilize signals free from noise, artifacts, and other distortions that could mislead the model to enhance the quality of signal measurements for accurate model training. Further, mislabeled data can lead to a poorly performing model. Therefore, quality control measures may be implemented to allow for the accuracy and reliability of training datasets.

At step 220, the method 200 proceeds with preprocessing the plurality of cardiorespiratory signal measurements in each patient record to ensure a common length with cardiac cycles and a common alignment with cardiac or respiratory cycles. As discussed earlier, aligning signals with cardiac and/or respiratory cycles may be beneficial because different sensors often operate at distinct sampling frequencies. Directly combining signals with varying lengths may lead to misalignment and misrepresentation of the temporal relationship between events in a cardiac cycle. Cardiorespiratory signals generally are inherently linked to the cardiac and/or respiratory cycle. Analyzing these signals in isolation or without proper alignment can obscure crucial information about their interdependence. Preprocessing the plurality of cardiorespiratory signal measurements may aid in future feature extraction and subsequent analysis.

At step 230, the method 200 comprises extracting features from preprocessed cardiorespiratory signal measurements. The raw cardiorespiratory signals (e.g., ECG, SCG) are generally complex waveforms containing a wealth of information. Feature extraction is the process of transforming raw signals into a set of representative features that capture information relevant to the task at hand (in this case, classifying cardiorespiratory conditions). These features are designed to be more informative and easier for the deep learning model to process than the raw signals themselves. Feature extraction often involves reducing the dimensionality of the data (e.g., summarizing the vast amount of information contained in the raw signals into a smaller set of meaningful features). This may simplify the learning task for the model and help prevent overfitting. It should be noted that step 230 is optional, as the preprocessed cardiorespiratory signal measurements can be forwarded directly to the deep learning model.

Feature extraction may also involve identifying and quantifying relevant characteristics from the preprocessed signals, such as temporal, spectral, spectrotemporal, statistical, and morphological features. Temporal features generally capture the timing and sequence of events in the signal, such as heart rate variability (HRV), beat-to-beat intervals (R-R intervals), or the duration of specific waveforms (e.g., QRS complex in ECG). These features can reveal abnormalities in heart rhythm or autonomic nervous system function. Spectral features generally analyze the frequency content of the signals, revealing information about different physiological processes. Examples include the power spectral density (PSD) of the SCG signal (which can reveal information about the contractility and stiffness of the heart), dominant frequency components, or the presence of specific frequency bands associated with particular conditions. Morphological features generally describe the shape and structure of the waveforms, such as the amplitude, slope, or curvature of specific segments in the signal. These features can be indicative of valve dysfunction, myocardial ischemia, or other cardiac abnormalities. Combined features that integrate information from multiple signals may also be generated. For example, a feature could combine HRV from the ECG signal with specific frequency components from the SCG signal to provide a more comprehensive assessment of cardiovascular function. By incorporating diverse features, the model can learn a broader range of patterns associated with different cardiorespiratory conditions.

A range of techniques can be employed for feature extraction, depending on the specific signal type and the desired information. Some common methods include time-domain analysis, frequency-domain analysis, time-frequency analysis, nonlinear analysis, and machine learning-based methods. Time-domain analysis examples include statistical measures (e.g., mean, variance), peak detection, and waveform duration analysis. Examples of frequency-domain analysis include Fourier transform, wavelet transform, and spectral entropy. Nonlinear analysis examples include Lyapunov exponents, fractal dimension, and entropy measures. Examples of time-frequency analysis include short-term Fourier transform, wavelet transform, and chirplet transform. Examples of machine learning-based methods include autoencoders and principal component analysis (PCA).

At step 240, the method 200 comprises training a deep learning model. This may include training the model on extracted features, the preprocessed cardiorespiratory signal measurements, or combinations thereof along with corresponding diagnosis to classify cardiorespiratory signals into categories corresponding to cardiorespiratory conditions. Deep learning models, a subset of artificial intelligence, are able to find patterns in large, complex datasets. In the case of cardiorespiratory monitoring, the dataset may be the collection of test subject records with their associated cardiorespiratory signals and diagnoses. As discussed, these features might include heart rate variability from ECG, specific vibration patterns from SCG, or changes in chest impedance.

A deep learning model may be trained on the extracted features and/or the preprocessed cardiorespiratory signal measurements and corresponding diagnoses to learn the complex patterns and relationships that distinguish different cardiorespiratory conditions. The specific architecture of the deep learning model can vary depending on the design choices and the desired performance. Some common architectures used in this context may include convolutional neural networks (CNNs), recurrent neural networks (RNNs), and hybrid models. CNNs may be particularly well-suited for analyzing signals with spatial or temporal patterns, such as ECG and SCG signals. CNNs can automatically learn to extract relevant features from the signals, reducing the reliance on manual feature engineering. RNNs are generally designed to handle sequential data, making them suitable for analyzing cardiorespiratory signals that evolve over time. RNNs can capture temporal dependencies and long-term patterns in the signals. Combining CNNs and RNNs, or other types of models, can offer a powerful way to leverage the strengths of different architectures and achieve superior performance in classifying complex cardiorespiratory signals. Thus, CNN or RNN deep learning models are generally adept at learning intricate patterns and relationships within data.

The training process may be considered supervised if the model is provided with the correct answers (e.g., the diagnoses) during training. In instances, the categories corresponding to the cardiorespiratory conditions may be indicated using a binary set of status groups comprising a normal cardiorespiratory status and an abnormal cardiorespiratory status. This binary classification scheme, where cardiorespiratory conditions are categorized into two status groups, may simplify the interpretation of results, allowing for quick assessments of overall cardiorespiratory health. Alternatively, or in conjunction with the binary approach, the cardiorespiratory conditions may be indicated as a list of potential medical diagnoses. These varied approaches may provide more detailed information to healthcare professionals, aiding in diagnosing and managing specific cardiorespiratory diseases. Hence, depending on the design and the training data, these categories can be broad (e.g., normal vs. abnormal) or more specific (e.g., atrial fibrillation, heart failure).

After step 240, the method 200 may end. Alternatively, method 200 may continue with step 250 (discussed infra). Thus, step 250 is optional, and method 200 may proceed from step 240 to end.

At step 250, the method 200 may further comprise evaluating performance of the trained deep learning model on a separate validation dataset. This may be achieved by testing the model on a separate validation dataset, which was not used during the training process. This step may improve the model in that the model can generalize to new, unseen data and provide a reliable estimate of its accuracy and effectiveness in real-world scenarios.

Deep learning models, particularly those with numerous parameters, may be susceptible to overfitting. This means the model might learn the specific patterns in the training data too well, performing excellently on the training data but poorly on new, unseen data. For a medical diagnostic tool, it may be useful that the model generalizes well, meaning it can accurately classify cardiorespiratory conditions in test subjects it hasn't encountered before. A separate validation dataset, distinct from the training dataset, may provide a way to assess the model's ability to generalize. This dataset may contain cardiorespiratory signal measurements and diagnoses from patients not included in the training process.

Evaluating a model's performance typically involves calculating various metrics, such as accuracy, sensitivity, specificity, precision, F1 score, and Area Under the Receiver Operating Characteristic Curve (AUROC). Accuracy generally refers to the overall percentage of correct predictions. Sensitivity (or recall) generally refers to the ability of a model to correctly identify positive cases (e.g., patients with a specific cardiorespiratory condition). Specificity generally refers to the ability of a model to correctly identify negative cases (e.g., healthy test subjects). Precision generally refers to the proportion of positive predictions that are actually true positives. F1 score signifies a balanced measure that combines precision and recall. AUROC is a measure of a model's ability to distinguish between different classes (e.g., different cardiorespiratory conditions).

A validation process may involve data splitting, model training, model evaluation, metric calculation, and iterative improvement. First, the original dataset may be split into two parts: a training dataset and a validation dataset. Typically, a majority of the data (e.g., 80%) is used for training, and a smaller portion (e.g., 20%) is used for validation. Then, the model may be trained on the training dataset, as previously described. The trained model may then be used to predict the diagnoses for test subjects in the validation dataset as part of model evaluation. For metric calculation, the model's predictions may be compared to the actual diagnoses in the validation dataset, and the relevant performance metrics may be calculated. Based on the evaluation results, the model architecture or training process may be refined and the evaluation repeated. This iterative process may continue until satisfactory performance of the validation dataset is achieved.

In another embodiment, a training process may involve initialization, data gathering, forward pass, error calculation, backpropagation, iteration, and validation. The model may start with random parameters (e.g., weights and biases). The extracted features from the training dataset, along with their corresponding diagnoses, may be fed into the model. The model may make predictions about the diagnoses based on the input features. The model's predictions may be compared to the actual diagnoses, and the error (e.g., loss) may be calculated. For backpropagation, the error may be used to update the model's parameters to reduce the error for future predictions. For iteration, the data gathering, forward pass, error calculation, and backpropagation steps may be repeated many times, with the model gradually improving its ability to classify the data accurately. During validation, the trained model may be tested on a separate validation dataset (not seen during training) to allow for it to generalize well to new data and safeguard it from simply memorizing training examples.

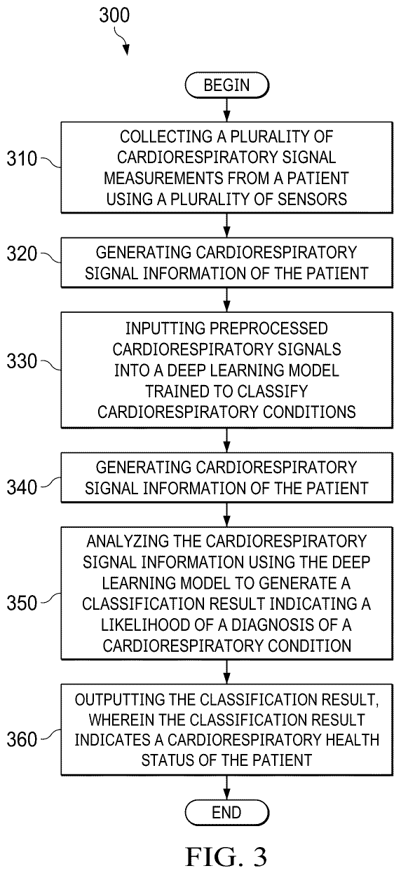

FIG. 3 illustrates a method 300 for monitoring cardiorespiratory conditions in a test subject using a multimodal cardiorespiratory monitor according to an embodiment of the disclosure. The method 300 utilizes a previously trained deep learning model, for example, deep learning model 170, to analyze instantaneous cardiorespiratory signals collected from the test subject and provide a classification result indicating the likelihood of various cardiorespiratory conditions.

At step 310, the method 300 includes collecting a plurality of cardiorespiratory signal measurements from a test subject using a plurality of sensors. As discussed previously, example cardiorespiratory monitoring devices often rely on a single signal, such as ECG or pulse oximetry, to assess cardiac and respiratory health. These single signals may not fully capture the complex interplay of various physiological parameters, leading to incomplete or inaccurate assessments.

Here, the cardiorespiratory signal measurements may be collected using various sensors integrated into the multimodal cardiorespiratory monitor (e.g., multimodal cardiorespiratory monitor system 100), such as ECG, SCG, GCG, PCG, pulse oximetry, body temperature, and chest impedance sensors (either individually or in combination). This multimodal approach may allow for a comprehensive assessment of the test subject's cardiorespiratory health, including electrical and mechanical aspects of cardiac function, as well as respiratory parameters and tissue characteristics. Simultaneously collecting cardiorespiratory signals may ensure that all signals are captured within the same cardiac and/or respiratory cycle cycle, allowing for a more accurate representation of the dynamic interactions between different physiological parameters. By collecting multiple signals simultaneously, subtle correlations and patterns that might be missed by single-signal devices may be captured. This can lead to earlier detection and more accurate diagnosis of a wider range of cardiorespiratory conditions, including those that manifest differently in various individuals.

At step 320, the method 300 further comprises preprocessing the plurality of cardiorespiratory signal measurements to ensure a common length with respect to an average cardiac and/or respiratory cycles and a common alignment with cardiac and/or respiratory cycles using an electrocardiogram (ECG) signal and/or a low frequency domponent of SCG as reference(s). It should be noted that the length of a cardiac cycle is variable and constantly changes. Accordingly, an average can be used to adjust for such variability. As discussed earlier, the alignment of signals with cardiac cycles may be beneficial because different sensors often operate at distinct sampling frequencies. For example, the ECG sensor might sample at 100 Hz while the SCG sensor samples at 50 Hz. Directly combining signals with varying lengths may lead to misalignment and misrepresentation of the temporal relationship between events in a cardiac cycle. Cardiorespiratory signals may naturally be linked to the cardiac cycle, which consists of distinct phases like systole (e.g., contraction) and diastole (e.g., relaxation). Analyzing these signals in isolation or without proper alignment could obscure information about their interdependence.

Subsequently extracting meaningful features from the signals may rely on consistent signal lengths and alignments with cardiac and/or respiratory phases. For instance, SCG or GCG features like systolic time intervals or diastolic durations are generally meaningful only when calculated from signals that are accurately synchronized with the cardiac cycle. A deep learning model, which is trained to recognize patterns in aligned and synchronized signals, may expect input data to have a consistent structure. Misaligned or unsynchronized signals could hinder the model's ability to learn and classify accurately.

An ECG signal is generally chosen as a reference for alignment due to its well-defined waveforms (e.g., P, QRS, T) corresponding to specific cardiac events. The prominent R-peaks in the ECG signal generally provide clear markers for the onset of each cardiac cycle. By aligning other cardiorespiratory signals (e.g., SCG, GCG) to the ECG R-peaks, signals can be synchronized to the same cardiac cycle, facilitating accurate comparison and analysis.

An SCG signal may also be employed to segment the signals into different phases of respiratory cycle. The respiratory cycle can be estimated using the low-frequency vibrations of the chest measured by SCG.

Preprocessing may involve multiple steps, such as signal filtering, ECG peak detection, signal segmentation, signal resampling, and signal alignment. Raw signals from all sensors may be filtered (e.g., through digital filters) to remove noise and artifacts, enhancing signal quality (e.g., signal-to-noise ratio). Different types of filters may be used, such as high-pass filters to remove baseline wander, low-pass filters to attenuate high-frequency noise, and notch filters to suppress powerline interference.

The filtered ECG signal may be analyzed to detect the characteristic R-peaks, which mark the onset of each cardiac cycle. For example, an algorithm like the Pan-Tompkins algorithm may be employed to detect R-peaks in the ECG signal more accurately (even in the presence of noise and artifacts). In another example, template matching or deep learning algorithms may be employed to detect R peaks. Other cardiorespiratory signals (e.g., SCG, GCG) may be segmented into individual cardiac cycles based on the identified ECG R-peaks (as previously discussed). If necessary, signals with different sampling rates may be resampled to a common frequency to allow for uniform length for subsequent analysis. All segmented signals may be aligned based on their respective cardiac cycles such that the corresponding features occur at the same relative time points.

At step 330, the method 300 comprises inputting preprocessed cardiorespiratory signals into a deep learning model trained to classify cardiorespiratory conditions. This step represents the transition from raw physiological data to the realm of artificial intelligence (AI), where the deep learning model may leverage its trained knowledge to interpret the signals and classify a test subject's cardiorespiratory status.

As discussed earlier, raw signals from multiple sensors undergo preprocessing to ensure they are clean, aligned, and synchronized. This prepares the signals for better analysis by the deep learning model. The deep learning model, as previously described, has already been trained on a large and diverse dataset of cardiorespiratory signals and corresponding diagnoses. It has learned to recognize patterns in the signals that are indicative of different cardiorespiratory conditions. The preprocessed signals are inputs that the deep learning model uses to make predictions. The model takes these signals and applies the knowledge it has learned during training to classify the test subject's cardiorespiratory status. As previously discussed, the specific architecture of the deep learning model (e.g., CNNs, RNNs) can vary depending on the design choices and the desired performance.

The preprocessed signals may be formatted into a suitable input shape for the deep learning model. This may involve converting the signals into numerical arrays or matrices that the model can efficiently process. In some cases, the preprocessed signals may be further transformed into a set of extracted features before being fed into the model. These features could be hand-crafted based on domain knowledge or learned automatically by the model during training. The input signals may be divided into batches to improve computational efficiency and enable parallel processing. Later, the deep learning model may process the input signals and generate classification results, as discussed. These results can be labeled in a binary (e.g., normal or abnormal) or be a list of potential diagnoses along with their respective probabilities or confidence scores.

At step 340, the method 300 comprises generating cardiorespiratory signal information of the test subject. This refers to the process of deriving insights and representations from the preprocessed signals, making them suitable for further analysis and classification by the deep learning model. This step generally aids in bridging the gap between raw sensor data and the higher-level understanding of the test subject's cardiorespiratory health.

As discussed earlier, the raw signals from the multiple sensors may be preprocessed to ensure they are clean, aligned, and synchronized. While these preprocessed signals are more amenable to analysis than raw data, they still contain a vast amount of information that should be distilled into a more concise and informative format. The generation of cardiorespiratory signal information typically involves extracting relevant features from the preprocessed signals. These features can be thought of as a summary of the more salient characteristics of the signals that are relevant for diagnosing cardiorespiratory conditions. The generated signal information serves as the input for the deep learning model. The model may have been trained to recognize patterns in these specific features and correlate them with different cardiorespiratory conditions.

By generating meaningful signal information, the deep learning model may provide interpretable results that can be used for clinical decision-making. This information may include not only the likelihood of specific conditions or their comorbidity but also the confidence levels associated with each diagnosis.

As discussed earlier, the specific type of information generated can vary depending on the design choices and the desired clinical outcomes. Some examples of cardiorespiratory signal information types include temporal, spectral, morphological, and combined features. Various signal processing and machine learning techniques can be used to generate cardiorespiratory signal information, including time-domain analysis, frequency-domain analysis, time-frequency analysis, nonlinear analysis, and machine learning-based methods.

At step 350, the method 300 comprises analyzing the cardiorespiratory signal information using the deep learning model to generate a classification result indicating a likelihood of a diagnosis of a cardiorespiratory condition or comorbidity of several cardiorespiratory conditions. This step may mark the culmination of the data collection and preprocessing phases, where the preprocessed cardiorespiratory signals and the extracted features are fed into the trained deep learning model to obtain a meaningful classification result.

Deep learning models, particularly artificial neural networks, have the ability to discern complex patterns within large datasets. In the context of medical diagnostics, these models can be trained to recognize subtle correlations between physiological signals and disease states, often exceeding the capabilities of traditional rule-based methods. The multimodal nature of the collected data, encompassing various cardiorespiratory signals, presents a multi-dimensional problem that deep learning may be uniquely suited to address. The model can analyze the complex interplay between different physiological parameters, identifying patterns that might be missed by examining individual signals in isolation.

The analysis process may involve various layers (e.g., input, hidden, output). The preprocessed cardiorespiratory signals and extracted features may be inputted into the input layer of the deep learning model. The model's hidden layers may process the input data through a series of complex mathematical operations, learning to identify relevant patterns and relationships. The final layer of the model may produce the classification result, which can be a binary label, a list of potential diagnoses, or a probability distribution over different conditions.

At step 360, the method 300 comprises outputting the classification result, in which the classification result indicates a cardiorespiratory health status of the test subject. A goal of cardiorespiratory monitors is to provide actionable insights into the test subject's health. This may be achieved by translating the complex analysis performed by the deep learning model into a clear and understandable output that can guide clinical decision-making or inform the test subject about their health status.

The classification result can be presented in various formats depending on the intended audience and the specific use case. For healthcare professionals, the output might be a detailed report with specific diagnoses, probabilities, and relevant clinical information. For individual users, the output might be a simplified indicator of overall cardiorespiratory health or a notification about potential risk factors, which might be sent to the user's doctor or health professional through text message or in a mobile, watch, or computer application.

In outputting the classification result, a binary classification approach may be utilized where the test subject's cardiorespiratory health status is indicated using two categories (e.g., normal and abnormal). This may provide a simple and intuitive way for users to understand the overall assessment of their cardiorespiratory health. Alternatively, or in conjunction with the binary classification approach, the cardiorespiratory health status may be provided as a list of potential medical diagnoses along with their respective probabilities. This may offer more detailed information to healthcare professionals, aiding in diagnosing and managing specific conditions. This may also allow healthcare professionals to assess the reliability of the classification and make informed decisions about further diagnostic tests or treatment plans by providing more nuanced interpretations of results, taking into account the uncertainty sometimes inherent in any diagnostic process.

The classification results may be further refined by including probabilities or confidence scores associated with each potential diagnosis. Confidence scores in this context may represent the model's level of certainty in its classification decision. In other words, they may indicate how sure the model is that a particular test subject has a particular cardiorespiratory condition or falls within a specific health status category (e.g., normal vs. abnormal). Several methods can be employed to calculate confidence scores. Some examples include SoftMax output, Ensemble methods, Bayesian deep learning, or calibration techniques. The confidence score can be presented as a simple numerical value between 0 and 1 (or 0% and 100%), where higher values indicate greater confidence. The confidence score can be visually represented using a bar graph, a color gradient, or other visual cues to make it easier for users to interpret. In addition to numerical values, the confidence score can be accompanied by qualitative descriptions, such as high confidence, moderate confidence, or low confidence, to provide a more intuitive understanding of the model's certainty.

These scores can be helpful for both healthcare professionals and test subjects, as they can provide a nuanced understanding of the model's predictions and help guide further diagnostic or treatment decisions. For example, high confidence scores can increase confidence in the model's diagnosis, while low confidence scores may warrant further investigation or additional tests. In cases where multiple conditions are predicted, confidence scores can help prioritize interventions based on the likely and concerning diagnoses. Further, by tracking changes in confidence scores over time, clinicians can monitor the progression or improvement of a cardiorespiratory condition and adjust treatment plans accordingly.

The output may be delivered through a user interface (e.g., user interface 180), which can be a display screen on a wearable device, a mobile app, or even auditory alerts. The design of the user interface may prioritize clarity, case of interpretation, and relevance to the user's needs.

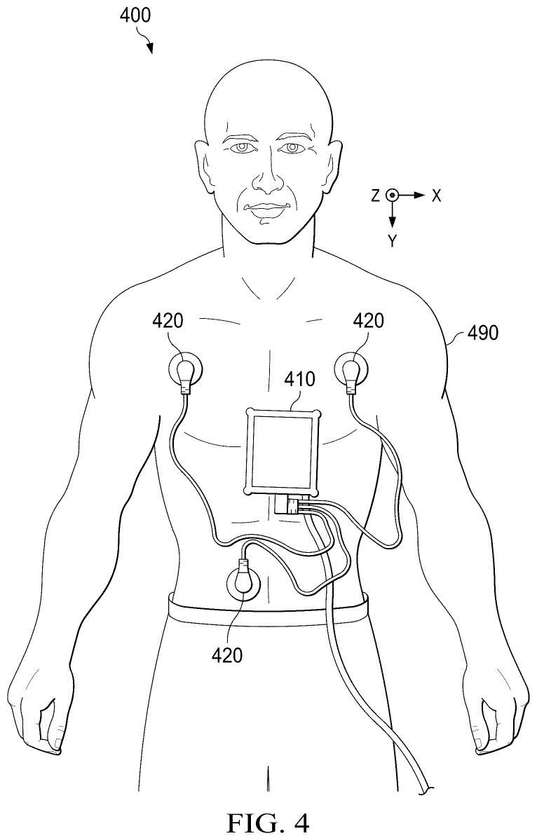

FIG. 4 is a diagram 400 depicting the placement of sensors on a human body for multimodal cardiorespiratory monitoring according to an embodiment of the disclosure. A multimodal cardiorespiratory signal gathering device 410 and sensors 420 may be placed on body 490. Sensors 420 may include ECG sensors, SCG sensors, GCG sensors, PCG sensors, pulse oximeters, temperature sensor, and chest impedance sensors.

As depicted, sensors 420 are a plurality of ECG sensors for measuring ECG signals. A relevant number of adhesive ECG electrodes (e.g., two to ten) are placed on the torso of body 490-under the right and left clavicle and the lower right side of the abdomen. These sensors 420 allow for the measurement of the electrical activity of the heart in a clinically relevant way. It should be noted that ECG electrodes may be placed anywhere on the chest that is clinically acceptable. Accordingly, the configuration shown should not be considered limiting. A pulse oximeter sensor (not depicted) may also be placed on the index finger of the right hand of body 490. This sensor is typically clipped onto a fingertip or earlobe. It uses light absorption principles to measure the oxygen saturation level in the blood.

Similarly, other sensors may be placed on body 490 to gather signal measurements. For example, such sensors can be included in signal gathering device 410. An SCG sensor may be a uni-axial, dual-axial, or tri-axial accelerometer sensor attached to the chest, typically at the sternum level. A tri-axial sensor measures vibrations in three axes (e.g., X, Y, and Z) caused by the mechanical activity of the heart and lung. A GCG sensor is usually placed adjacent to the SCG sensor on the chest. It measures the torsional vibrations of the chest surface caused by the heart's twisting motion. A PCG sensor is a microphone-based sensor that is placed anywhere on the chest such as near the apex of the heart, or above the cardiac valves to provide a more pronounced sound of the opening and closing of these valves. It captures the subtle sounds produced by the heart during its pumping cycle and lung during the respiration cycle. Chest impedance sensors may be two additional electrodes placed on the chest, strategically spaced apart from the ECG electrodes used for signal measurement. A small alternating current may be passed through these electrodes, and the resulting impedance may be measured. This provides information about changes in the underlying tissue, such as fluid accumulation in the lungs. A body temperature sensor may also be placed on the body at any clinically acceptable location, such as in the signal gathering device 410. This provides changing temperature values that can also be fed back into the machine learning model.

The specific placement locations for each sensor on a body (e.g., body 490) may vary slightly based on individual anatomy and comfort or the type of medical information that is desired. The sensors should generally be securely attached to enhance the accuracy and consistency of measurements. Using hypoallergenic materials and proper skin preparation can reduce potential skin irritation or discomfort. For long-term monitoring, it may be beneficial to prioritize user comfort and ease of use when designing sensor placement. By strategically placing these sensors on the body, the multimodal cardiorespiratory monitor can capture a wide range of physiological signals that contribute to a comprehensive assessment of cardiorespiratory health.

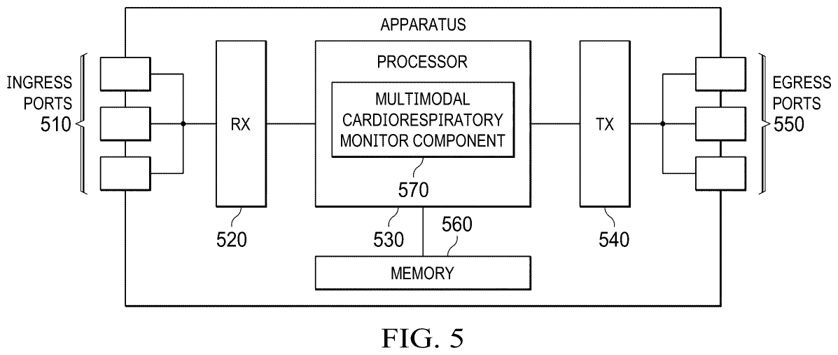

FIG. 5 is a schematic diagram of an apparatus 500 according to an embodiment of the disclosure. The apparatus 500 may implement the disclosed embodiments. The apparatus 500 comprises ingress ports 510 and a receiver unit (RX) 520 to receive data; a processor 530, or logic unit, baseband unit, or central processing unit (CPU), to process the data; a transmitter unit (TX) 540 and egress ports 550 to transmit the data; and a memory 560 to store the data. The apparatus 500 may also comprise optical-to-electrical (OE) components, electrical-to-optical (EO) components, or radio frequency (RF) components coupled to the ingress ports 510, the RX 520, the TX 540, and the egress ports 550 to provide ingress or egress of optical signals, electrical signals, or RF signals.

The processor 530 is any combination of hardware, middleware, firmware, or software. The processor 530 comprises any combination of one or more CPU chips, graphical processing unit (GPU) chips, cores, field-programmable gate array (FPGAs), application-specific integrated circuit (ASICs), or digital signal processor (DSPs). The processor 530 communicates with the ingress ports 510, the RX 520, the TX 540, the egress ports 550, and the memory 560. The processor 530 comprises a multimodal cardiorespiratory monitor component 570, which implements the disclosed embodiments. The inclusion of the multimodal cardiorespiratory monitor component 570 therefore provides a substantial improvement to the functionality of the apparatus 500 and effects a transformation of the apparatus 500 to a different state. Alternatively, the memory 560 stores the multimodal cardiorespiratory monitor component 570 as instructions, and the processor 530 executes those instructions.

The memory 560 comprises any combination of disks, tape drives, solid-state drives or other technologies. The apparatus 500 may use the memory 560 as an overflow data storage device to store programs when the apparatus 500 selects those programs for execution and to store instructions and data that the apparatus 500 reads during execution of those programs. The memory 560 may be volatile or non-volatile and may be any combination of read-only memory (ROM), random-access memory (RAM), ternary content-addressable memory (TCAM), static RAM (SRAM), or other technologies.

A computer program product may comprise computer-executable instructions that are stored on a computer-readable medium and that, when executed by a processor, cause an apparatus to perform any of the embodiments. The non-transitory medium may be the memory 560, the processor may be the processor 530, and the apparatus may be the apparatus 500.

There may also be other embodiments and/or aspects of the disclosure that are not depicted. For example, an embodiment of a deep learning model architecture may include an input layer, convolutional layer, recurrent layer, feature concatenation, fully connected layers, and classification output layer.

In an input layer of the deep learning model architecture, multiple input branches, each corresponding to a different type of preprocessed cardiorespiratory signal (e.g., ECG, SCG, GCG, PCG, pulse oximetry, body temperature, chest impedance) may receive signal data as a sequence of numerical values representing the amplitude of the signal at different time points. Each branch may include one or more convolutional layers to extract local patterns and features from the signals. These layers may apply filters to the input data, capturing specific shapes and patterns that are relevant for classification. Different filter sizes and numbers of filters can be used to capture features at different scales. Further, one or more recurrent layers, such as Long Short-Term Memory (LSTM) or Gated Recurrent Unit (GRU) layers, may be used to capture temporal dependencies and long-term patterns in the signals. These layers may process the input data sequentially, maintaining a hidden state that allows them to remember information from previous time steps. The outputs from the different branches (either directly from the input layer or after passing through convolutional or recurrent layers) may be concatenated into a single feature vector. This feature vector may represent a combined representation of the different cardiorespiratory signals. One or more fully connected layers may process the concatenated feature vector, further integrating the information from different modalities. These layers may learn complex relationships between the features and the corresponding diagnoses. The final layer of the model may produce the classification result, which can be binary or multi-class. In the case of binary classification, a single output neuron with a sigmoid activation function, indicating the probability of the test subject having a normal or abnormal cardiorespiratory status, may be the ease. Multi-Class Classification may involve multiple output neurons with a SoftMax activation function, indicating the probabilities of different cardiorespiratory conditions (e.g., atrial fibrillation, heart failure).

The specific architecture of the deep learning model may be customized based on the characteristics of the dataset and the desired performance. The number and types of layers, the size of the filters, the number of neurons in the fully connected layers, and the activation functions may be adjusted to optimize the model's performance. The model may be trained using various optimization algorithms, such as stochastic gradient descent (SGD) or Adam, and loss functions, such as cross-entropy loss.

While several embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

In addition, techniques, systems, subsystems, and methods described and illustrated in the various embodiments as discrete or separate may be combined or integrated with other systems, components, techniques, or methods without departing from the scope of the present disclosure. Other items shown or discussed as coupled may be directly coupled or may be indirectly coupled or communicating through some interface, device, or intermediate component whether electrically, mechanically, or otherwise. Other examples of changes, substitutions, and alterations are ascertainable by one skilled in the art and may be made without departing from the spirit and scope disclosed herein.

What is claimed is:

1. A method comprising:
receiving a training dataset comprising a plurality of test subject records, wherein each patient record comprises:
a plurality of cardiorespiratory signal measurements from a patient; and
a corresponding diagnosis of one or more cardiorespiratory conditions for the patient;
preprocessing the plurality of cardiorespiratory signal measurements in each patient record to ensure a common length with cardiorespiratory cycles and a common alignment with cardiorespiratory cycles;
training a deep learning model on cardiorepiratory data and corresponding diagnosis to classify cardiorespiratory signals into categories corresponding to cardiorespiratory conditions.

2. The method of claim 1, wherein the cardiorepiratory data includes extracted features from preprocessed cardiorespiratory signal measurements, the preprocessed cardiorespiratory signal measurements, or combinations thereof.

3. The method of claim 1, wherein the cardiorespiratory signal measurements are performed on one or more of:
one or more electrocardiogram (ECG) signals;
one or more seismocardiogram (SCG) signals;
one or more gyrocardiogram (GCG) signals;
a phonocardiogram (PCG) signal;
a pulse oximetry signal;
a body temperature signal; and
a chest impedance signal.

4. The method of claim 1, wherein the categories corresponding to the cardiorespiratory conditions are indicated using a binary set of status groups comprising a normal cardiorespiratory status and an abnormal cardiorespiratory status.

5. The method of claim 1, wherein the categories corresponding to the cardiorespiratory conditions are indicated as a list of potential medical diagnoses.

6. The method of claim 1, wherein the cardiorespiratory conditions are a comorbidity of cardiorespiratory conditions, and wherein the categories corresponding to the comorbidity of cardiorespiratory conditions are indicated as a list of potential medical diagnoses.

7. The method of claim 1, wherein the training dataset further comprises demographic information for each patient, alcohol consumption habits, sugar consumption habits, physical activities, or combinations thereof.

8. The method of claim 1, further comprising evaluating performance of the trained deep learning model on a separate validation dataset.

9. A method comprising:
collecting a plurality of cardiorespiratory signal measurements from a patient using a plurality of sensors;
preprocessing the plurality of cardiorespiratory signal measurements to ensure a common length with respect to cardiorespiratory cycles and a common alignment with cardiorespiratory cycles using an electrocardiogram (ECG) signal and a seismocardiogram (SCG) signal as references; inputting cardiorepiratory data into a deep learning model trained to classify cardiorespiratory conditions;
analyzing the cardiorepiratory data using the deep learning model to generate a classification result indicating a likelihood of a diagnosis of a cardiorespiratory condition; and
outputting the classification result, wherein the classification result indicates a cardiorespiratory health status of the patient.

10. The method of claim 9, wherein collecting a plurality of cardiorespiratory signal measurements from the patient is performed simultaneously.

11. The method of claim 9, wherein the cardiorespiratory health status is indicated using a binary set of status groups comprising a normal cardiorespiratory status and an abnormal cardiorespiratory status.

12. The method of claim 9, wherein the cardiorespiratory health status comprises a list of potential medical diagnoses.

13. The method of claim 9, wherein the classification result includes a confidence score associated with each diagnosis of a cardiorespiratory condition.

14. An apparatus comprising:
a plurality of sensors configured to collect a plurality of cardiorespiratory signal measurements;
a memory configured to store a deep learning model trained on a dataset comprising a plurality of test subject records, each record including a plurality of cardiorespiratory signal measurements and a corresponding diagnosis of a cardiorespiratory condition; and
a processor operatively coupled to the plurality of sensors and the memory, wherein the processor is configured to:
preprocess the plurality of cardiorespiratory signal measurements to ensure a common length with respect to cardiorespiratory cycles and a common alignment with cardiorespiratory cycles using an ECG signal and a seismocardiogram (SCG) signal as a reference;
input cardiorepiratory data into the deep learning model;
analyze the cardiorepiratory data using the deep learning model to generate a classification result indicating a likelihood of a diagnosis of a cardiorespiratory condition; and
output the classification result, wherein the classification result further indicates a cardiorespiratory health status.

15. The apparatus of claim 14, wherein the cardiorespiratory signal measurements are performed on one or more of:
one or more electrocardiogram (ECG) signals;
one or more seismocardiogram (SCG) signals;
one or more gyroscopicardiogram (GCG) signals;
a phonocardiogram (PCG) signal;
a pulse oximetry signal; and
a chest impedance signal.

16. The apparatus of claim 14, wherein the plurality of sensors are configured to collect the plurality of cardiorespiratory signal measurements simultaneously.

17. The apparatus of claim 14, wherein the cardiorespiratory health status is indicated using a binary set of status groups comprising a normal cardiorespiratory status and an abnormal cardiorespiratory status.

18. The apparatus of claim 14, wherein the cardiorespiratory health status comprises a list of potential medical diagnoses or a comorbidity of multiple conditions.

19. The apparatus of claim 14, wherein the memory further stores instructions for segmenting the plurality of cardiorespiratory signals based on cardiorespiratory cycles.

20. The apparatus of claim 14, further comprising a user interface for receiving input and for showing the output including sensor signals or classification results.
